(19) United States
(12) Patent Application Publication
(10) Pub. No.: US 2020/0108018 A1
(43) Pub. Date: Apr. 9, 2020

(54) CANNABIS PRODUCTS AND INDUSTRIAL METHODS FOR PRODUCTION THEREOF

(71) Applicant: BUZZELET DEVELOPMENT AND TECHNOLOGIES LTD, Or-Akiva (IL)

(72) Inventors: Yuri SHADURIN, Binyamina (IL); Meir ARIEL, Kazir (IL); Aharon M. EYAL, Jerusalem (IL); Looz MILAY, Pardes Hanna-Karkur (IL)

(73) Assignee: BUZZELET DEVELOPMENT AND TECHNOLOGIES LTD, Or-Akiva (IL)

(21) Appl. No.: 16/319,074

(22) PCT Filed: Jul. 18, 2017
(86) PCT No.: PCT/IB2017/054339

§ 371 Date: Jan. 18, 2019

Related U.S. Application Data
(60) Provisional application No. 62/363,910, filed on Jul. 19, 2016.

(51) Int. Cl. A61K 9/20; A61K 36/185; A24B 15/167; A24C 5/39; A24C 5/02; A24D 1/02; A24D 1/18

(52) U.S. Cl. CPC A61K 9/2095; A61K 36/185; A61K 9/20; A24B 15/167; A61K 2236/333; A24C 5/02; A24D 1/02; A24D 1/18; A61K 2236/15; A24C 5/393

(57) ABSTRACT

Cannabis products and industrial methods for production thereof. A homogeneous composition comprising cannabinoid-containing comminuted cannabis plant material, where at least 70 weight % of the comminuted cannabis plant material is of a size greater than 0.3 millimeter and less than 4.2 millimeters; the composition comprises at least 1% by weight moisture and less than 20% by weight moisture; and the cannabinoid concentration in a 1 gram fraction of at least 10 gram sample of said homogeneous composition, is within 15% of the cannabinoid concentration in a separate 1 gram fraction of said 10 gram sample.

### CROSS-REFERENCE TO RELATED APPLICATIONS

The present application claims the benefit of U.S. Provisional Application Ser. No. 62/363,910 filed Jul. 19, 2016, the disclosure of which is expressly incorporated by reference herein in its entirety.

### BACKGROUND OF THE INVENTION

The therapeutic effects of cannabis are known for thousands of years, but only recently the mechanisms of operation are being uncovered.

The main active components are named cannabinoids. So far, more than 100 cannabinoids were discovered. Cannabis plants contain multiple cannabinoids, sometimes dozens of them. The most known cannabinoids are tetrahydrocannabinol (THC) and cannabidiol (CBD). Others include cannabigerol (CBG), cannabichromene (CBC) tetrahydrocannabivarin (THCV) Cannabidivarin (CBDV) and cannabinol (CBN). Cannabinoids are found in various parts of the cannabis plant. Typically, their concentration is greatest in the flower (the bud), particularly in the trichomes.

In the cannabis plant, these cannabinoids are mostly in their acid form. For most therapeutic applications, cannabinoids are heat treated to effect thermal decarboxylation of the cannabinoids, which is sometimes referred to as activation.

Cannabis plants also include other components, e.g. terpenes (terpenoids) and falvenoids. There are claims that terpenes impact the mechanism of operation of the cannabinoids. Known cannabis terpenes include pinene, limonene, linalool, caryophyllene, myrcene, humulene, borneol, eucalyptol, terpineol, nerolidol, phytol, geraniol, bisabolol and camphene.

There are hundreds of cannabis plant strains, which differ in many aspects, including their cannabinoids content. For example, some are high on THC (more than 20 wt %), while others have high CBD/THC ratios. There are also major differences in the terpenes content.

Therapeutic cannabis is used in various forms, e.g. smoking, vaporizers, oils, ointments, pills, cookies, drinks, sprays, including nasal ones, etc. In vaporizers, plant material, oil or other forms are heated to a temperature and for a time suitable for decarboxylation and evaporation and the patient inhales the formed vapors.

Therapeutic use requires high standard of manufacture, high product quality, accuracy in dosing and repeatability, so that the patient gets each time the compositions found most suitable for her or him.

Regulations in many countries are changing these days to allow therapeutic use of cannabis and the number of treated patients is already many millions. There is therefore desire for industrial production generating products at best quality standards.

Developing industrial methods for cannabis products is complicated by various aspects, including the large number of strains with major composition differences between them. There is also the need to avoid losses of important components either by evaporation or via thermal degradation, including during heat decarboxylation.

An example for such difficulties is the production of cannabis oil via extraction of the plant material. High yields are required for commercial effectiveness and extraction should maintain the ratio between the important active components. Additionally on the one hand, extraction kinetics is accelerated by reducing plant particles size, but on the other, too small size leads to caking of the material and therefore inefficient contact. Additionally, comminution efficiency, extraction yield and extract composition are strongly dependent on strain properties and plant material moisture content, which in turn is affected by time from harvest and storing conditions.

Also important are the difficulties found in cigarettes production. More than one half of the patient still get their cannabis via smoking and they need cigarettes that are uniform in composition throughout and burn smoothly. Content should not change from one cigarette to another. Flow of air should be convenient. Technologies developed for tobacco cigarettes are not suitable for cannabis cigarettes because of the difference in nature between the cannabis and tobacco and because of the more strict requirements of the therapeutic treatment. Cannabis cigarettes are typically hand made because of these difficulties.

Vaporizers are used by patients that do not want to smoke, cannot smoke or ones that stay in hospitals or other institutes where smoking is forbidden. They need their cannabis in a form that easily fits into the vaporizer they use (much variety), avoids liter and doesn't require a very steady hand.

Hence, there is a strong need for cannabis products that are reliable, accurate and suitable for use by treated patients. There is also a need for method to produce such product on industrial scale and at industrial production rate.

### SUMMARY OF THE INVENTION

A homogeneous composition comprising cannabinoid-containing comminuted cannabis plant material is described, including at least 70 weight % of the comminuted cannabis plant material is of a size greater than 0.3 millimeter and less than 4.2 millimeters; the composition comprises at least 1% by weight moisture and less than 20% by weight moisture; and the cannabinoid concentration in a 1 gram fraction of at least 10 gram sample of said homogeneous composition, is within 15% of the cannabinoid concentration in a separate 1 gram fraction of said 10 gram sample.

Additional embodiments include: the homogeneous composition described above where at least 70 weight % of the cannabis plant material in said composition is derived from cannabis flower; the homogeneous composition described above having a bulk density greater than 0.02 gram/milliliter; the homogeneous composition described above having a bulk density less than 0.6 gram/milliliter; a cigarette containing the homogeneous composition described above; and a vaporizer cartridge containing the homogeneous composition described above.

A homogeneous cannabinoid-containing cannabis tablet is also described, where said tablet moisture content is greater than 1% by weight and less than 20% by weight; said tablet bulk density is at least 0.1 gram/milliliter and less than 1.2 gram/milliliter; and the cannabinoid concentration in a fraction of said tablet is within 15% of the cannabinoid concentration in a separate fraction of said tablet. The homogeneous cannabis tablet described above where said tablet comprises at least 70% weight cannabis plant material is also described.

A method for producing a homogeneous composition comprising comminuted cannabis plant material, is also described including providing cannabis plant material comprising at least one cannabinoid and moisture, wherein said moisture content is less than 20 weight %; maintaining said provided cannabis plant material at a temperature of less than 0° C. for at least 1 hour to form frozen plant material; comminuting said frozen cannabis plant material by pressing said plant material against a mesh screen with a sieve size smaller than 4.2 millimiters to form an expelled homogeneous composition comprising comminuted cannabis plant material; and collecting said expelled, homogeneous composition.

Additional embodiments include: the method described above comprising providing a first cannabis plant material whereby a first collected expelled, homogeneous composition is formed, providing a second cannabis plant material whereby a second collected expelled, homogeneous composition is formed, and blending said first collected expelled, homogeneous composition with said second collected expelled, homogeneous composition to form a homogeneous blend composition; the method of claim 10, further comprising analyzing a sample of said first collected expelled, homogeneous composition, analyzing a sample of said second collected expelled, homogeneous composition and blending said first composition and said second composition in a proportion calculated to form a homogeneous blend composition of pre-determined cannabinoid content; the method described above where at least 70 weight % of said provided cannabis plant material is derived from cannabis flower; the method described above where said screen pore size is at least 0.3 millimeter; the method described above where said provided plant material comprises moisture at more than 1 weight %; the method described above where said homogeneous composition is characterized in that on sampling at least two separate 1 gram fractions of at least 10 gram sample of said homogeneous composition, the concentrations of said cannabinoid in the at least two 1 gram fractions are within 15% of each other; the method described above where the bulk density of said homogeneous composition is greater than 0.02 gram/milliliter; the method described above where the bulk density of said homogeneous composition is less than 0.6 gram/milliliter; the method described above further comprising adding said homogeneous composition to a cigarettes tube or a cigarette paper; the method described above further comprising (i) providing said homogeneous composition; (ii) introducing said homogeneous composition into a feed vessel; (iii) providing a cigarette tube or a cigarette paper; and (iv) transferring said homogeneous composition from said feed vessel into said provided tube or onto said paper to form a cigarette, and where said transferring comprises vibrating the feed vessel at a rate of 0 to 200 vibrations per minute; the method described above where said transferring said homogeneous composition from said feed vessel comprises transferring said homogeneous composition onto a tray and transferring said homogeneous composition from said tray into said tube or onto said paper; the method described above where said transferring said homogeneous composition from said feed vessel to said tray is conducted by means of a screw conveyor; the method described above further comprising blending said provided homogeneous composition with an additive to form a homogeneous mixture, which homogeneous mixture is transferred into said cigarette tube or onto said paper; the method described above where said additive comprises an additional cannabis plant material and/or a non-cannabis plant material; and the method described above where said provided cigarette tube is cone shaped; the method described above where said provided cigarette tube and/or said provided cigarette paper is derived from hemp.

A cannabis cigarette produced according to the method described above is also described where the cigarette contains a top fraction and a bottom fraction and the concentration of said cannabinoid in a 0.1 gram sample from the top fraction of the cigarette is within 15% of cannabinoid concentration in a 0.1 gram sample from the bottom fraction of the cigarette. A batch of cannabis cigarettes produced according to the method described above is also described where the content of said cannabinoid in one third of the batch of the cigarettes is within 15% of the cannabinoid content in a separate third of the batch of the cigarettes.

Additional embodiments include: a method described above further comprising contacting said collected homogeneous cannabis plant material with a liquid extractant for at least 1 minute to form cannabinoid-depleted plant material and a cannabinoid-enriched extractant, separating said cannabinoid-depleted plant material from said cannabinoid-enriched extractant and separating at least a fraction of said extractant from said cannabinoid-enriched extractant to form a cannabinoid-enriched extract; the method described above further comprising blending at least a fraction of said cannabinoid-depleted plant material with at least a fraction of said collected homogeneous cannabis plant material; the method described above where said cannabinoid-depleted plant material contains extractant, and further comprising separating said extractant from said cannabinoid-depleted plant material to form desolventized cannabinoid-depleted plant material; the method described above further comprising mixing at least a fraction of said desolventized cannabinoid-depleted plant material with at least a fraction of said collected homogeneous cannabis plant material; the method described above further comprising pressing said collected homogeneous composition to form a homogeneous cannabis tablet; the method described above where the tablet has a bulk density of at least 0.1 gram/milliliter; the method described above where the tablet has a bulk density of less than 1.2 gram/milliliter; the method described above where the concentration of said cannabinoid in a fraction of said homogeneous cannabis tablet is within 15% of the cannabinoid concentration in a separate fraction of the tablet.

An apparatus for the production of cannabis cigarettes is also described, comprising (i) a cigarette tubes holder, (ii) a feed vessel designed to contain a homogeneous composition comprising comminuted cannabis plant material and containing at least one opening and (iii) at least one first conveyor connected between said vessel opening and said holder. An apparatus described above is also described further comprising a second conveyer designed to transfer homogeneous composition from said vessel, through said opening, onto said first conveyor connected between said vessel and said first conveyor.

A homogeneous micronic composition is also described comprising cannabinoid-containing comminuted cannabis plant material, where at least 70 weight % of the comminuted cannabis plant material is of a size less than 0.05 millimeter; the composition comprises at least 1% by weight moisture and less than 20% by weight moisture; and the cannabinoid concentration in a 1 gram fraction of at least 10 gram sample of said homogeneous composition, is within 15% of the cannabinoid concentration in a separate 1 gram fraction of said 10 gram sample.

Additional embodiments include: the homogeneous micronic composition described above where at least 50 weight % of the cannabinoid in said homogeneous composition is a decarboxylated form; and the homogeneous micronic composition described above where at least 70 weight % of the cannabis plant material in said composition is derived from cannabis flower. A vaporizer cartridge containing the homogeneous micronic composition described above is also described.

A method for producing a homogeneous micronic composition is also described comprising comminuted cannabis plant material, comprising providing cannabis plant material comprising at least one cannabinoid and moisture, wherein said moisture content is less than 20 weight %; maintaining said provided cannabis plant material at a temperature of less than 0° C. for at least 1 hour to form frozen plant material; and milling said cannabis plant material to particle size of less than 0.05 millimeter.

Additional embodiments include: the method described above where at least 50 weight % of the cannabinoid in said provided cannabis plant material is in a decarboxylated form; the method described above comprising providing a first cannabis plant material whereby a first homogeneous micronic composition is formed, providing a second cannabis plant material whereby a second homogeneous micronic composition is formed, and blending said first homogeneous micronic composition with said second homogeneous micronic composition to form a homogeneous micronic blend composition; the method described above where said homogeneous micronic composition is characterized in that on sampling at least two separate 1 gram fractions of at least 10 gram sample of said composition, the concentrations of said cannabinoid in the at least two 1 gram fractions are within 15% of each other; and the method described above, further comprising granulating said homogeneous micronic composition.

### BRIEF DESCRIPTION OF THE DRAWING

FIG. 1 shows a schematic flow plan of an embodiment of a method for production of cannabis products

FIG. 2 shows a schematic representation of an embodiment example of cannabis cigarette filling machine

### DETAILED DESCRIPTION OF THE INVENTION

The particulars shown herein are by way of example and for purposes of illustrative discussion of the various embodiments of the present invention only and are presented in the cause of providing what is believed to be the most useful and readily understood description of the principles and conceptual aspects of the invention. In this regard, no attempt is made to show details of the invention in more detail than is necessary for a fundamental understanding of the invention, the description making apparent to those skilled in the art how the several forms of the invention may be embodied in practice.

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise.

Unless indicated otherwise, percent is weight percent.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Additional advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention, as claimed.

According to a first aspect, provided is a homogeneous composition comprising cannabinoid-containing comminuted cannabis plant material, wherein (i) at least 70 weight % of the comminuted cannabis plant material is of a size greater than 0.3 millimeter and less than 4.2 millimeter; (ii) the composition comprises at least 1% by weight moisture and less than 20% by weight moisture; and (iii) the cannabinoid concentration in a 1 gram fraction of at least 10 gram sample of said homogeneous composition, is within 15% of the cannabinoid concentration in a separate 1 gram fraction of said 10 gram sample.

According to an embodiment, said homogeneous composition comprises at least two cannabinoids, at least three, at least four or at least five. According to an embodiment, the concentration of each cannabinoid in said composition is at least 10 parts per million (ppm). As known in the art, cannabinoids have an acid form and a non-acid form (which is also referred to as decarboxylated form, since it can be generated by decarboxylating the acid form). The acid form is indicated herein by the letter (a) at the end of the cannabinoid acronym, e.g. tetrahydrocannabiniolic acid is indicated as THCa, while the decarboxylated form is THC. As used herein, unless specified otherwise, the term cannabinoid and the name of the cannabinoid refers to both the acid form and the decarboxylated form. For example, tetrahydrocannabinol refers to THC and/or THCa. According to an embodiment, said cannabinoid is selected from the group consisting of tetrahydrocannabiniol in acid or decarboxylated form (THCa or THC, respectively), cannabidiol in acid or decarboxylated form (CBDa or CBD, respectively), cannabigerol in acid or decarboxylated form (CBGa or CBG, respectively), cannabichromene in acid or decarboxylated form (CBCa or CBC, respectively) tetrahydrocannabivarin in acid or decarboxylated form (THCVa or THCV, respectively), Cannabidivarin in acid or decarboxylated form (CBDVa or CBDV respectively) and cannabinol in acid or decarboxylated form (CBNa or CBN, respectively) and combinations thereof.

According to an embodiment said homogeneous composition comprises at least one terpene, at least two, at least three, at least four or at least five terpenes. The term “terpene”, as used herein, refers to terpenes and/or terpenoids. According to an embodiment, said terpene is selected from the group consisting of pinene, limonene, linalool, caryophyllene, myrcene, humulene, borneol, eucalyptol, terpineol, nerolidol, phytol, geraniol, bisabolol, camphene, beta-amyrin, thujone, citronellol, pulegone, cycloartenol, isomers thereof and combinations thereof.

According to an embodiment at least 70 wt % of the composition comprises cannabis plant material, at least 75 wt %, least 80 wt %, least 85 wt %, least 90 wt % or at least 95 wt %. According to an embodiment at least 70 wt % of the cannabis plant material in said composition is derived from cannabis flower (also referred to as cannabis bud), at least 75 wt %, least 80 wt %, least 85 wt %, least 90 wt % or at least 95 wt %. As used herein, derived from cannabis flower refers to being cannabis flower or being a product or processing cannabis flower, e.g. drying and/or comminuting a cannabis flower. According to an embodiment, at least a fraction of said cannabis plant material in said composition comprises dried plant material.

According to an embodiment said composition comprises comminuted cannabis plant material, which is a collection of small particles. According to an embodiment at least 70 wt % of the particles, at least 80%, at least 90% or at least 95%, are of a size greater than 0.3 millimeter (mm), greater than 0.5 mm, greater than 0.7 mm, greater than 0.9 mm, greater than 1.1 mm or greater than 1.3 mm. As used herein, particle size refers to the largest dimension of said particles. According to an embodiment at least 70 wt % of the particles, at least 80%, at least 90% or at least 95% are of a size less than 4.2 mm, less than 4.0 mm, less than 3.8 mm, less than 3.6 mm less than 3.4 mm, less than 3.2 mm, less than 3.0 mm, less than 2.6 mm or less than 2.4.

According to an embodiment said composition comprises moisture. As used herein, the term moisture refers to water contained in the plant material or wetting it. According to an embodiment, moisture content can be determined by weight loss on drying. According to an embodiment said composition comprises at least 1 wt % moisture; at least 3 wt %, at least 5 wt %, at least 7 wt %, at least 9 wt %, or at least 11 wt %. According to an embodiment said composition comprises less than 20 wt % moisture, less than 18 wt %, less than 16 wt %, or less than 14 wt %.

Any amount of said composition is suitable. According to an embodiment, the amount of said composition is at least 100 gram (gr), at least 1 kilogram (Kg), at least 10 Kg, at least 100 Kg, or at least 1000 Kg.

According to an embodiment, said composition, is homogeneous. As used herein, the term homogeneous refers to having a similar content in various samples of the composition. According to an embodiment, a similar content means at least one of a similar size distribution and a similar concentration of at least one major component, at least two, at least three, at least four or at least five major components. As used herein, the term major component refers to a component comprising at least 1% of said composition.

According to an embodiment, the size of at least 70 weight %, at least 80% or at least 90% of the comminuted cannabis plant material in a 1 gram fraction of at least 10 gram sample of said homogeneous composition, is within 15% of the size of at least 70 weight %, at least 80% or at least 90% of the comminuted cannabis plant material in a separate 1 gram fraction of said 10 gram sample, within 13%, within 11%, within 9%, within 7%, within 5%, within 3%, within 2% or within 1%.

According to an embodiment, said major component is a cannabinoid and the cannabinoid concentration in a 1 gram fraction of at least 10 gram sample of said homogeneous composition, is within 15% of the cannabinoid concentration in a separate 1 gram fraction of said 10 gram sample within 13%, within 11%, within 9%, within 7%, within 5%, within 3%, within 2% or within 1%. According to an embodiment said cannabinoid comprises tetrahydrocannabinol (THC). According to another embodiment said cannabinoid comprises cannabidiol (CBD).

According to an embodiment, said homogeneous composition comprises at least 0.5% terpene and the terpene concentration in a 1 gram fraction of at least 10 gram sample of said homogeneous composition, is within 15% of the terpene concentration in a separate 1 gram fraction of said 10 gram sample within 13%, within 11%, within 9%, within 7%, within 5%, within 3%, within 2% or within 1%.

According to an embodiment, the bulk density of said composition is greater than 0.02 gram/milliliter (gr/ml), greater than 0.04 gr/ml, greater than 0.06 gr/ml, greater than 0.08 gr/ml, greater than 0.1 gr/ml or greater than 0.12 gr/ml. According to an embodiment, the bulk density of said composition is less than 0/6 gr/ml, less than 0.5 gr/ml, less than 0.4 gr/ml, less than 0.3 gr/ml, less than 0.25 gr/ml or less than 0.2 gr/ml.

Further provided is a product containing said homogeneous composition. Further provided is a cannabis cigarette comprising said homogeneous composition. According to an embodiment, said cannabis cigarette burns smoothly during smoking and as a result, according to an embodiment, said cannabis cigarette doesn't require re-lighting during smoking.

According to an embodiment, said cigarette contains at least 0.1 gr of said homogeneous material, at least 0.3 gr or at least 0.5 gr. According to an embodiment, said cigarette comprises less than 2 gr of said homogeneous material, less than 1.5 gr, less than 1.2 gr or less than 1 gr. According to an embodiment, said cigarette contains a top fraction and a bottom fraction and the concentration of a major component in a 0.1 gram sample from the top fraction of the cigarette is within 15% of the concentration of said major component in a 0.1 gram sample from the bottom fraction of the cigarette, within 13%, within 11%, within 9%, within 7%, within 5%, within 3%, within 2% or within 1%. According to an embodiment, said cigarette contains a top fraction and a bottom fraction and the concentration of said cannabinoid in a 0.1 gram sample from the top fraction of the cigarette is within 15% of cannabinoid concentration in a 0.1 gram sample from the bottom fraction of the cigarette, within 13%, within 11%, within 9%, within 7%, within 5%, within 3%, within 2% or within 1%.

Further provided is a vaporizer cartridge containing said homogeneous composition.

Further provided is a homogeneous cannabis tablet, wherein (i) said tablet moisture content is greater than 1 wt % and less than 20 wt %; (ii) said tablet bulk density is at least 0.1 gr/ml and less than 1.2 gr/ml; and (iii) the cannabinoid concentration in a fraction of said tablet is within 15% of the cannabinoid concentration in a separate fraction of said tablet, within 13%, within 11%, within 9%, within 7%, within 5%, within 3%, within 2% or within 1%.

According to an embodiment said cannabis tablet comprises moisture. According to an embodiment said cannabis tablet comprises at least 1 wt % moisture; at least 3 wt %, at least 5 wt %, at least 7 wt %, at least 9 wt %, or at least 11 wt %. According to an embodiment said cannabis tablet comprises less than 20 wt % moisture, less than 18 wt %, less than 16 wt %, or less than 14 wt %.

According to an embodiment, the bulk density of said cannabis tablet is greater than 0.1 gr/ml, greater than 0.3 gr/ml, greater than 0.5 gr/ml, greater than 0.6 gr/ml, greater than 0.7 gr/ml, greater than 0.8 gr/ml or greater than 0.85 gr/ml. According to an embodiment, the bulk density of said cannabis tablet is less than 1.2 gr/ml, less than 1.1 gr/ml, less than 1.05 gr/ml, less than 1.0 gr/ml, less than 0.95 gr/ml or less than 0.90 gr/ml.

According to an embodiment, said cannabis tablet, is homogeneous. As used herein, the term homogeneous refers to having similar content in various fractions of the tablet. According to an embodiment, similar content is determined by chemical analysis of a major component. Thus, according to an embodiment the concentration of a cannabinoid in a fraction of said homogeneous cannabis tablet is within 15% of the cannabinoid concentration in a separate fraction, within 13%, within 11%, within 9%, within 7%, within 5%, within 3%, within 2% or within 1%. According to an embodiment, said cannabinoid comprises THC and/or CBD. According to an embodiment, said tablet comprises at least 0.5% terpene and the terpene concentration in a fraction of said tablet is within 15% of the terpene concentration in a separate fraction of said tablet, within 13%, within 11%, within 9%, within 7%, within 5%, within 3%, within 2% or within 1%.

According to an embodiment at least 70 wt % of said cannabis tablet comprises cannabis plant material, at least 75 wt %, least 80 wt %, least 85 wt %, least 90 wt % or at least 95 wt %. According to an embodiment at least 70 wt % of said cannabis tablet is derived from cannabis plant flower, at least 75 wt %, least 80 wt %, least 85 wt %, least 90 wt % or at least 95 wt %.

According to an embodiment, the weight of said tablet is between 0.1 gr and 2 gr, between 0.2 gr and 1.5 gr or between 0.25 gr and 1 gr.

Further provided is a method for producing a homogeneous, comminuted, cannabis plant material composition, comprising (i) providing cannabis plant material comprising moisture, wherein moisture content is less than 20 wt %; (ii) keeping said provided cannabis plant material at a temperature of less than 0° C. for at least 1 hour to form frozen plant material; (iii) comminuting said frozen cannabis plant material by pressing against a screen with a pore size of less than 4.2 mm to form expelled homogeneous composition comprising comminuted cannabis plant material; and (iv) collecting said expelled, homogeneous composition.

According to an embodiment at least 70 wt % of said provided cannabis plant material is derived from cannabis plant flower, at least 75 wt %, least 80 wt %, least 85 wt %, least 90 wt % or at least 95 wt %.

According to an embodiment said provided cannabis plant material comprises less than 20 wt % moisture, less than 18 wt %, less than 16 wt %, or less than 14 wt %. According to an embodiment said providing cannabis plant material comprises at least 1 wt % moisture; at least 3 wt %, at least 5 wt %, at least 7 wt %, at least 9 wt %, or at least 11 wt %.

According to an embodiment said provided cannabis plant material is kept at a temperature of less than 0 degrees Celsius (° C.) for at least 1 hour (hr) to form said frozen plant material According to an embodiment said provided cannabis plant material in kept at a temperature of less than 0° C., less than minus 5° C., less than minus 10° C., less than minus 15° C. or less than minus 20° C. According to an embodiment said provided cannabis plant material is kept at a said temperature for at least 1 hour (hr) to form frozen plant material, for at least 2 hr, at least 4 hr, at least 6 hr, at least 8 hr, at least 10 hr, at least 12 hr, at least 14 hr or at least 16 hr.

According to an embodiment said frozen cannabis plant material is comminuted by pressing against a screen with a pore size of less than 4.2 mm to form expelled homogeneous composition comprising comminuted cannabis plant material. According to an embodiment said screen comprises at least one wire. According to an embodiment said wire diameter is at least 0.1 mm, at least 0.2 mm or at least 0.3 mm. According to an embodiment, said screen is shaped as a drum or a fraction of the drum. According to an embodiment, pressing is done by an oscillating unit within said drum. According to an embodiment, oscillation is conducted at a rate between 30 and 500 rounds per minute (RPM), between 50 and 450 RPM or between 100 and 400 RPM.

According to an embodiment, screen pore size is less than 4.0 mm, less than 3.8 mm, less than 3.6 mm, less than 3.4 mm, less than 3.2 mm or less than 3.0 mm According to an embodiment, screen pore size is at least 0.3 mm, at least 0.6 mm, at least 0.8 mm, at least 1.0 mm, at least 1.2 mm, at least 1.4 mm, at least 1.6 mm, at least 1.8 mm or at least 2.0 mm.

According to an embodiment, said method further comprises collecting said expelled, homogeneous composition. Any form of collecting is suitable.

According to an embodiment, said method comprises forming said homogeneous composition from two or more strains. According to an embodiment, said method further comprises blending collected expelled, homogeneous composition generated from provided one cannabis plant strain with homogeneous composition generated from another provided cannabis plant strain. The ratio between the amounts of blended compositions can be of any ratio.

According to an embodiment, said method comprises providing a first cannabis plant material whereby a first collected expelled, homogeneous composition is formed, providing a second cannabis plant material whereby a second collected expelled, homogeneous composition is formed, and blending said first collected expelled, homogeneous composition with said second collected expelled, homogeneous composition to form a homogeneous blend composition. According to an embodiment, said method further comprises analyzing a sample of said first collected expelled, homogeneous composition, analyzing a sample of said second collected expelled, homogeneous composition and blending said first composition and said second composition in a proportion calculated to form a homogeneous blend composition of pre-determined cannabinoid content.

Any rate of production is applicable. According to an embodiment, said homogeneous composition is produced at a rate of at least 1 Kg/hr, at least 10 Kg/hr, at least 100 Kg/hr or at least 1000 Kg/hr.

According to an embodiment, said produced homogeneous composition, is characterized by having a similar content in various samples of the composition. According to an embodiment, a similar content means at least one of a similar size distribution and a similar concentration of at least one major component, at least two, at least three, at least four or at least five major components. As used herein, the term major component refers to a component comprising at least 1% of said composition.

According to an embodiment, said produced homogeneous composition, is characterized by that the size of at least 70 weight %, at least 80% or at least 90% of the comminuted cannabis plant material in a 1 gram fraction of at least 10 gram sample of said homogeneous composition, is within 15% of the size of at least 70 weight %, at least 80% or at least 90% of the comminuted cannabis plant material in a separate 1 gram fraction of said 10 gram sample, within 13%, within 11%, within 9%, within 7%, within 5%, within 3%, within 2% or within 1%.

According to an embodiment, said major component is a cannabinoid and said produced homogeneous composition, is characterized by that the cannabinoid concentration in a 1 gram fraction of at least 10 gram sample of said homogeneous composition, is within 15% of the cannabinoid concentration in a separate 1 gram fraction of said 10 gram sample within 13%, within 11%, within 9%, within 7%, within 5%, within 3%, within 2% or within 1%. According to an embodiment said cannabinoid comprises tetrahydrocannabinol (THC).

According to another embodiment said cannabinoid comprises cannabidiol (CBD).

According to an embodiment, said homogeneous composition comprises at least 0.5% terpene and said produced homogeneous composition, is characterized by that the terpene concentration in a 1 gram fraction of at least 10 gram sample of said homogeneous composition, is within 15% of the terpene concentration in a separate 1 gram fraction of said 10 gram sample within 13%, within 11%, within 9%, within 7%, within 5%, within 3%, within 2% or within 1%.

According to an embodiment, the bulk density of said composition is greater than 0.02 gr/ml, greater than 0.04 gr/ml, greater than 0.06 gr/ml, greater than 0.08 gr/ml, greater than 0.1 gr/ml or greater than 0.12 gr/ml. According to an embodiment, the bulk density of said composition is less than 0.6 gr/ml, less than 0.5 gr/ml, less than 0.4 gr/ml, less than 0.3 gr/ml, less than 0.25 gr/ml or less than 0.2 gr/ml.

According to an embodiment, said method further comprises producing cannabis cigarettes comprising said homogeneous composition. Producing cannabis cigarettes present different challenges than those of producing tobacco cigarettes due to the difference in nature between the cannabis plant material and tobacco. Cannabis cigarettes tend to irregular burning and to inconvenience of use. Packing and homogeneity are highly important. In order to resolve those, cannabis cigarettes are typically hand-made. Industrial production requires a method that enables high-rate machine manufacture, still at a quality reached in hand-made cigarettes. Additionally, handling large volumes of cannabis plant material typically leads to stratification in the material and therefore to cigarettes of varying content and/or varying composition. Industrial production should also maintain uniformity within each cigarette, avoiding regions with higher cannabinoids content compared to others. The inventors have discovered that these challenges are addressed by the method of the present invention, comprising adding said homogeneous composition to a cigarettes tube or a cigarette paper.

Thus, according to an embodiment, cannabis cigarettes production comprises (i) providing said homogeneous composition (ii) introducing said homogeneous composition into a feed vessel; (iii) providing a cigarette tube or a cigarette paper; and (iv) transferring said homogeneous composition from said feed vessel into said provided tubes or onto said paper to form a cigarette, wherein said transferring comprises vibrating the feed vessel at a rate of 0 to 200 vibrations per minute. According to an embodiment, said transferring is done with no vibration of the feed vessel. According to an embodiment, said transferring involves vibrating said feed vessel at a rate of up to 200 vibrations per minute.

Any feed vessel is suitable. Any form of introducing said homogeneous composition into said feed vessel is suitable, e.g. batch-wise or continuous. According to an embodiment, the amount of homogeneous composition in said feed vessel at a given time is less than 100 Kg, less than 50 Kg, less than 20 Kg or less than 10 Kg. According to an embodiment, the amount of homogeneous composition and vessel dimensions are selected so that the level of homogeneous composition there is less than 50 centimeter (cm), less than 40 cm, less than 30 cm, less than 20 cm, less than 15 cm or less than 10 cm. According to an embodiment, said homogeneous composition is introduced into said feed vessel at a rate of at least 1 Kg per hour, at least 5 Kg per hour or at least 10 Kg per hour.

According to an embodiment, the size of said cigarette tube or said cigarette paper is selected to contain at least 0.1 gr of said homogeneous material, at least 0.2 gr, at least 0.3 gr, at least 0.4 gr or at least 0.5 gr. According to an embodiment, the size of said cigarette tube or said cigarette paper is selected to contain less than 2.5 gr of said homogeneous material, less than 2.0 gr, less than 1.5 gr, less than 1.2 gr or less than 1.0 gr. According to an embodiment, said provided cigarette tubes are cone shaped. According to an embodiment, said provided cigarette tube and/or said provided cigarette paper is derived from hemp, e.g. said provided cigarette paper comprises hemp fibers

According to an embodiment provided cigarette tube and/or said provided cigarette paper comprise a spacer, according to an embodiment the spacer is a filter, according to an embodiments the spacer is a filter derived from hemp.

According to an embodiment, said homogeneous composition is transferred from said feed vessel into said provided cigarette tube or onto said provided cigarette paper to form said cigarettes. According to an embodiment, vibration of said feed vessel is avoided or minimized According to an embodiment, said transferring is conducted without vibrating said feed vessel. According to an embodiment, said transferring uses vibration at a rate of less than 200 vibrations per minute, less than 180, less than 160, less than 140, less than 120, less than 100, less than 80, less than 60, less than 40 or less than 20.

According to an embodiment, said transferring said homogeneous composition from said feed vessel into said provided cigarette tube or onto said provided cigarette paper comprises the use of a screw conveyor.

According to an embodiment, said transferring from said feed vessel into said tube, or onto said provided cigarette paper, comprises transferring said homogeneous composition from said feed vessel onto a tray and transferring said homogeneous composition from said tray into said tubes. According to an embodiment, said tray has a slope, e.g. a slope of 45 degrees or of 30 degrees. According to an embodiment, said transferring said homogeneous composition from said feed vessel onto said tray comprises the use of a screw conveyor. According to an embodiment, said filled tubes are vibrated for less than 1 minute for compaction.

According to an embodiment, the amount of homogeneous composition on said tray and the tray dimensions are selected so that the level of homogeneous composition on said tray is less than 10 cm less than 8 cm, less than 6 cm, less than 4 cm or less than 2 cm.

According to an embodiment said tray is vibrated. According to an embodiment said tray is vibrated at a rate of less than 200 vibrations per minute.

According to an embodiment, said transferring said homogeneous composition from said feed vessel into said provided cigarette tube or onto said provided cigarette paper is conducted while the provided cigarette tube or the provided cigarette paper has a filter and the transferring of the homogeneous composition is done over the filter of the provided cigarette tube or the provided cigarette paper, e.g. above it. According to an embodiment the provided cigarette tube or provided cigarette paper is folded after the transferring of the homogeneous composition in or onto the provided cigarette tube or provided cigarette paper.

According to an embodiment, the method further comprises blending said provided homogeneous composition with an additive to form a homogeneous mixture, which homogeneous mixture is transferred into said cigarette tubes or onto said cigarette paper. According to an embodiment, said additive comprises an additional cannabis plant material. According to an embodiment, said additive comprises a homogeneous composition generated according to the method of the present invention, by providing a different cannabis plant material. According to an embodiment, said additive comprises cannabinoids-depleted cannabis plant material, e.g. as formed by extraction of cannabis plant material. According to an embodiment, said additive comprises non-tobacco, non-cannabis smokeable plant material. According to an embodiment, said additive is selected from the group consisting of flavoring compounds, aroma compounds, terpenes, flavonoids and mixtures thereof. According to an embodiment, said blending said provided homogeneous composition with an additive is conducted before said transfer into said feed vessel.

According to an embodiment, said additive is selected from the group consisting of flavoring compounds, aroma compounds, terpenes, flavonoids and mixtures thereof and the method comprises diluting said additive in a diluent to form a solution or a suspension and applying said solution or suspension on said homogeneous composition to form sprayed composition. According to an embodiment, said method further comprises evaporating at least a fraction of the diluent from the sprayed composition, e.g. by exposing said sprayed composition to an elevated temperature. According to an embodiment, said diluent is selected from ethanol, water and mixtures thereof.

According to an embodiment, further provided is a cannabis cigarette produced according to said method of the present invention. According to an embodiment, said cigarette contains a top fraction and a bottom fraction and the concentration of said cannabinoid in a 0.1 gram sample from the top fraction of the cigarette is within 15% of cannabinoid concentration in a 0.1 gram sample from the bottom fraction of the cigarette, within 13%, within 11%, within 9%, within 7%, within 5%, within 3%, within 2% or within 1%.

According to an embodiment, further provided is a batch of cannabis cigarettes produced according to said method, e.g. 100 cigarettes, 500 cigarettes, 1000 cigarettes, 5000 cigarettes, or 10000 cigarettes, and the content of said cannabinoid in one of said cigarettes is within 15% of the cannabinoid content in a separate cigarette, within 13%, within 11%, within 9%, within 7%, within 5%, within 3%, within 2% or within 1%. According to an embodiment, said cannabinoid comprises THC and/or CBD.

According to an embodiment, further provided is a batch of cannabis cigarettes produced according to said method, e.g. 100 cigarettes, 500 cigarettes, 1000 cigarettes, 5000 cigarettes, or 10000 cigarettes, and the content of said cannabinoid in one third of the batch of the cigarettes is within 15% of the cannabinoid content in a separate third of the batch of the cigarettes, within 13%, within 11%, within 9%, within 7%, within 5%, within 3%, within 2% or within 1%. According to an embodiment, said cannabinoid comprises THC and/or CBD.

According to an embodiment, said method, further comprises contacting said homogeneous cannabis plant material with a liquid extractant for at least 1 minute to form cannabinoid-depleted plant material and a cannabinoid-enriched extractant, separating said cannabinoid-depleted plant material from said cannabinoid-enriched extractant and separating at least a fraction of said extractant from said cannabinoid-enriched extractant to form a cannabinoid-enriched extract.

According to an embodiment, said contacting is for at least 2 minutes, at least 4 minutes, at least 6 minutes, at least 8 minutes or at least 10 minutes. According to an embodiment, said extractant is selected from the group consisting of ethanol, ethanol-water mixtures, isopropanol, butane, butene, super-critical CO₂, sub-critical CO₂, near-critical CO₂, CO₂ mixtures with co-solvents and mixtures thereof.

According to an embodiment, said method further comprises separating extractant and cannabis extract out of said separated cannabinoid-enriched extractant. According to an embodiment, said separating extractant and cannabis extract comprises evaporation of the extract, at least partially.

According to an embodiment, said method further comprises dewaxing the cannabinoid-enriched extractant, whereby dewaxed extract is formed. According to an embodiment, said dewaxing comprises winterizing, e.g. cooling and filtering out precipitated waxes. According to an embodiment, said method comprises partially separating extractant out of said separated cannabinoid-enriched extractant, de-waxing the formed solution and then removing the rest of the diluent.

According to an embodiment, said method further comprises heating said extract or said dewaxed extract to a temperature greater than 80° C. for at least 1 minute, whereby cannabinoids contained therein decarboxylate and a decarboxylated extract is formed.

According to an embodiment, said method further comprises mixing at least a fraction of said cannabinoid-depleted plant material with at least a fraction of said collected homogeneous cannabis plant material, whereby a plant material mixture is formed. According to an embodiment, said method comprises transferring said plant material mixture into a cigarette tube or onto a cigarette paper to form a cigarette. Further provided are cannabis cigarettes comprising said plant material mixture.

According to an embodiment, said method comprises providing cannabis plant material derived from a first cannabis strain, freezing it to form a frozen material and comminuting said frozen material to form a homogeneous plant material derived from a first strain according to the method of the present invention. According to said embodiment, said method further comprises providing cannabis plant material derived from a second cannabis strain, contacting it with a liquid extractant to form cannabinoid-depleted plant material derived from said second strain and blending said homogeneous plant material derived from a first strain with said cannabinoid-depleted plant material derived from said second strain whereby a plant material mixture is formed. According to an embodiment, said method comprises transferring said plant material mixture into a cigarette tube or onto a cigarette paper to form a cigarette. Further provided are cannabis cigarettes comprising said plant material mixture.

According to an embodiment, said cannabinoid-depleted plant material contains extractant and the method further comprises separating extractant from said cannabinoid-depleted plant material to form desolventized cannabinoid-depleted plant material. According to an embodiment, said method further comprises mixing at least a fraction of said desolventized cannabinoid-depleted plant material with at least a fraction of said collected homogeneous cannabis plant material whereby a desolventized plant material mixture is formed. According to an embodiment, said method comprises transferring said desolventized plant material mixture into a cigarette tube or onto a cigarette paper to form a cigarette. Further provided are cannabis cigarettes comprising said desolventized plant material mixture.

According to an embodiment, said method further comprises pressing said collected homogeneous cannabis plant material to form a homogeneous cannabis tablet. According to an embodiment, said pressing is conducted in a tablet-making machine. According to an embodiment, said method comprises producing at least 10 tablets per minute, at least 100, at least 1000 or at least 10,000.

According to an embodiment at least 70 wt % of each said formed cannabis tablet comprises cannabis plant material, at least 75 wt %, least 80 wt %, least 85 wt %, least 90 wt % or at least 95 wt %. According to an embodiment at least 70 wt % of each said formed cannabis tablet comprises cannabis plant material derived from cannabis flowers, at least 75 wt %, least 80 wt %, least 85 wt %, least 90 wt % or at least 95 wt %.

According to an embodiment said formed cannabis tablet comprises moisture. According to an embodiment said formed cannabis tablet comprises at least 1 wt % moisture; at least 3 wt %, at least 5 wt %, at least 7 wt %, at least 9 wt %, or at least 11 wt %. According to an embodiment said formed cannabis tablet comprises less than 20 wt % moisture, less than 18 wt %, less than 16 wt %, or less than 14 wt %.

According to an embodiment, the bulk density of said formed cannabis tablet is greater than 0.1 gram/milliliter (gr/ml), greater than 0.3 gr/ml, greater than 0.5 gr/ml, greater than 0.6 gr/ml, greater than 0.7 gr/ml, greater than 0.8 gr/ml or greater than 0.85 gr/ml. According to an embodiment, the bulk density of said formed cannabis tablet is less than 1.2 gr/ml, less than 1.1 gr/ml, less than 1.05 gr/ml, less than 1.0 gr/ml, less than 0.95 gr/ml or less than 0.90 gr/ml.

According to an embodiment, said formed cannabis tablet, is homogeneous. According to an embodiment the concentration of cannabinoid in a fraction of said homogeneous cannabis tablet is within 15% of the cannabinoid concentration in a separate fraction, within 13%, within 11%, within 9%, within 7%, within 5%, within 3%, within 2% or within 1%. According to an embodiment, said cannabinoid comprises THC and/or CBD.

According to an embodiment, said tablet comprises at least 0.5% terpene and the terpene concentration in a fraction of said tablet is within 15% of the terpene concentration in a separate fraction of said tablet, within 13%, within 11%, within 9%, within 7%, within 5%, within 3%, within 2% or within 1%.

According to an embodiment, further provided is a batch of at least 10 homogeneous cannabis tablets produced according to said method, at least 100 or at least 1000, wherein cannabinoid content in one tablet is within 15% of the cannabinoid content in a separate tablet, within 13%, within 11%, within 9%, within 7%, within 5%, within 3%, within 2% or within 1%. According to an embodiment, further provided is a batch of homogeneous cannabis tablets produced according to said method, wherein cannabinoid content in one third of the tablets is within 15% of the THC content in a separate third of the tablets, within 13%, within 11%, within 9%, within 7%, within 5%, within 3%, within 2% or within 1%.

According to an embodiment, the weight of said tablet is at least 0.05 gr, at least 0.1 gr or at least 0.15 gr. According to an embodiment, the weight of said tablet is less than 1 gr, less than 0.8 gr, less than 0.6 gr or less than 0.4 gr. According to an embodiment, the cannabinoid content in said tablet is at least 2 milligrams (mg), at least 5 mg, at least 10 mg or at least 15 mg. According to an embodiment, the cannabinoid content in said tablet is less than 50 mg, less than 40 mg, less than 30 mg, or less than 20 mg.

According to an embodiment, the method further comprises heating said cannabis tablet to a temperature greater than 100° C., whereby said cannabinoid decarboxlates and/or evaporate. According to an embodiment, said heating decarboxylation and evaporation are conducted in a cannabis vaporizer.

According to a fourth aspect, provided is a system for the production of cannabis cigarettes, comprising a cigarette tubes holder, a feed vessel designed to contain a homogeneous composition comprising comminuted cannabis plant material and comprising at least one opening and at least one first conveyor connecting between said vessel opening and said holder. According to an embodiment, said first conveyor comprises a tray. According to an embodiment, said opening is at an elevation higher than said holder and said tray is positioned in a slope. According to an embodiment, said system further comprises a second conveyer designed to transfer homogeneous composition from said vessel, through said opening, onto said conveyor. According to an embodiment, said second conveyer comprises a screw conveyer.

### DETAILED DESCRIPTION OF THE DRAWING

In order to understand the invention and to see how it may be carried out in practice, embodiments will now be described, by way of non-limiting example only, with reference to the accompanying figures. In the figures, identical and similar structures, elements or parts thereof that appear in more than one figure are generally labeled with the same or similar references in the figures in which they appear. Dimensions of components and features shown in the figures are chosen primarily for convenience and clarity of presentation and are not necessarily to scale.

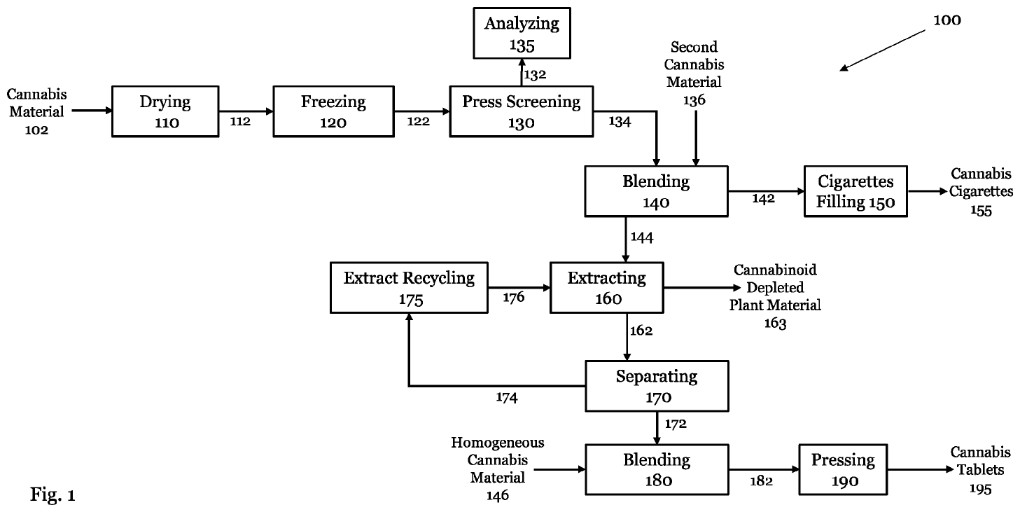

Reference is now made to FIG. 1 which is a schematic flow plan of an embodiment of a method for production of cannabis products according to an exemplary embodiments of the invention indicated generally as 100.

In the depicted exemplary embodiments, cannabis plant material (102) is dried in moisture controlled environment (110) to produce cannabis plant material with moisture level lower than 20% (112). The cannabis plant material with moisture level lower than 20% (112) is kept at a temperature of less than 0° C. (120) for at least 1 hour to form frozen plant material (122) and is then comminuting by pressing against a screen (130) to form expelled homogeneous composition (134), the expelled homogeneous composition (132) can be analyzed (135) to determine active materials content.

The expelled homogeneous composition (134) can be blended at (140) with second cannabis material (136) to form homogeneous blend composition (142) and (144). A fraction of the homogeneous blend composition (142) is sent to Cigarette production (150) to generate cannabis cigarettes (155).

A fraction of the homogeneous blend composition (144) is contacted (160) with liquid extractant (176) to form cannabinoid-depleted plant material (163) and a cannabinoid-enriched extractant (162). The cannabinoid-enriched extractant (162) is further treated (170) to separate at least a fraction of the extractant to form a cannabinoid-enriched extract (172) and a separated extractant (174). The separated extractant (174) can be treated (175) to re-generate liquid extractant (176).

The cannabinoid-enriched extract (172) is mixed (180) with homogeneous cannabis material (146) to form a plant material mixture (182) The homogeneous cannabis material (146) can be blended homogeneous composition (142), (144) or other blended homogeneous composition. The plant material mixture (182) is further pressed (190) to produce cannabis tablets (195).

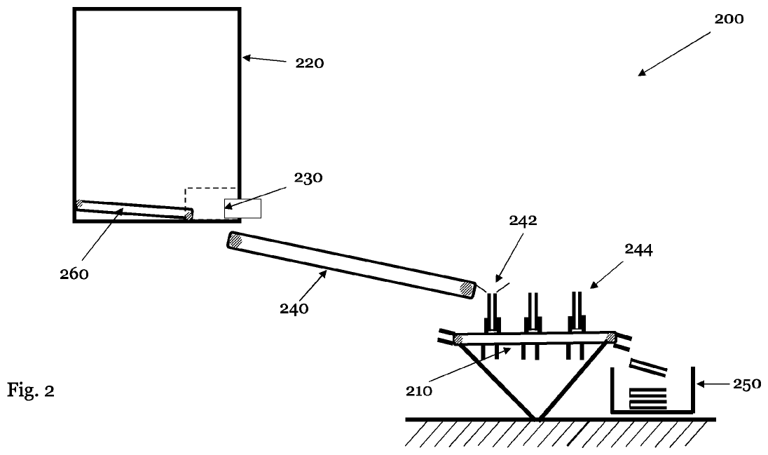

Reference is now made to FIG. 2 which shows a cannabis cigarette producing apparatus according to some exemplary embodiments of the invention indicated generally as 200.

The apparatus for the production of cannabis cigarettes, depicted in FIG. 2 comprises (i) a cigarette tubes (242) and (244) holder (210); (ii) a feed vessel (220) designed to contain a homogeneous composition comprising comminuted cannabis plant material, which feed vessel contains at least one opening (230), and (iii) at least one first conveyor (240) connected between said vessel opening and said holder. According to an embodiment, the apparatus further comprises a second conveyer (260) designed to transfer homogeneous composition from said vessel (220), through said opening (230), onto said first conveyor (240) connected between said vessel (220) and said first conveyor (240). According to an embodiment, the apparatus further comprises a vessel for collecting produced cigarettes (250).

Further provided is a homogeneous micronic composition comprising cannabinoid-containing comminuted cannabis plant material, wherein (i) at least 70 weight % of the comminuted cannabis plant material is of a size less than 0.05 millimeter (mm); (ii) the composition comprises at least 1% by weight moisture and less than 20% by weight moisture; and (iii) the cannabinoid concentration in a 1 gram fraction of at least 10 gram sample of said homogeneous composition, is within 15% of the cannabinoid concentration in a separate 1 gram fraction of said 10 gram sample. According to an embodiment, the comminuted cannabis plant material is of a size less than 0.05 mm, less than 0.03 mm, less than 0.01 mm or less than 0.005 mm.

According to an embodiment, at least 50 weight % of the cannabinoid in said homogeneous composition is in a decarboxylated form, at least 60%, at least 70%, at least 80% or at least 90%.

According to an embodiment, at least 70 weight % of the cannabis plant material in said composition is derived from cannabis flower, at least 80% or at least 90%.

Further provided is a vaporizer cartridge containing said homogeneous micronized composition.

Also provided is a method for producing a homogeneous micronic composition comprising comminuted cannabis plant material, comprising (i) providing cannabis plant material comprising at least one cannabinoid and moisture, wherein said moisture content is less than 20 weight %; (ii) maintaining said provided cannabis plant material at a temperature of less than 0° C. for at least 1 hour to form frozen plant material; and (iii) milling said cannabis plant material to particle size of less than 0.05 millimeter, less than 0.03 mm, less than 0.01 mm or less than 0.005 mm According to an embodiment, at least 50 weight % of the cannabinoid in said provided cannabis plant material is in a decarboxylated form, at least 60%, at least 70%, at least 80% or at least 90%.

According to an embodiment, said method comprises providing a first cannabis plant material whereby a first homogeneous micronic composition is formed, providing a second cannabis plant material whereby a second homogeneous micronic composition is formed, and blending said first homogeneous micronic composition with said second homogeneous micronic composition to form a homogeneous micronic blend composition.

According to an embodiment, said formed homogeneous micronic composition is characterized in that on sampling at least two separate 1 gram fractions of at least 10 gram sample of said composition, the concentrations of said cannabinoid in the at least two 1 gram fractions are within 15% of each other.

According to an embodiment, said method further comprising granulating said homogeneous micronic composition.

### EXAMPLES

### Examples 1-9: Production of Cannabis Comminuted Homogeneous Composition

Flowers of a cannabis plant strain were dried to moisture content of 14%. The dried flowers were kept in a freezer at a temperature of minus 20° C. for 24 hours. The frozen flowers were comminuted, as such, without warming, by pressing against a screen with a pore size of 2 mm. The expelled comminuted material was collected and allowed to warm to (plus) 20° C. Then a sample of the expelled material was taken and its bulk density was determined. The results are summarized in the following table.

TABLE

| Example 1 | Strain | Producer | Bulk density (gr/ml) |
|---|---|---|---|
| Example 2 | Magen | IMC, Israel | 0.140 |
| Example 3 | Dairy Queen | IMC, Israel | 0.162 |
| Example 4 | Pandora's Box | IMC, Israel | 0.154 |
| Example 5 | White | Seach, Israel | 0.143 |
| Example 6 | Pink | Seach, Israel | 0.135 |
| Example 7 | White Widow | Candoc, Israel | 0.132 |
| Example 8 | Jack Herer | Candoc, Israel | 0.132 |
| Example 9 | Can2 | Candoc, Israel | 0.140 |

### Example 10: Production of an Extract

10 gr of comminuted material prepared according to the previous examples were introduced into a beaker along with 100 ml of ethanol 95 wt %. The beaker was shaken for 10 minutes. Then the solid matter was filtered away from the solution. The filtered solid material was dried in an oven at 60° C. to form cannabinoid-depleted plant material. The liquid was introduced into a rotavapor beaker and the extractant was evaporated out at a temperature of 35° C. and at reduced pressure. The extract remaining in the rotavapor beaker was analyzed. It's cannabinoids (combined) content was greater than 80%.

### Example 11: Production of a Cannabis Tablet

10 gr of comminuted material prepared according to the Examples 1-9 were introduced into a tablet-making machine. 50 tablets were formed. The tablets sink when put in 95% ethanol solution and float when put in water.

1-46. (canceled)

47. A homogeneous composition comprising cannabinoid-containing comminuted cannabis plant material, wherein
(i) at least 70 weight % of the comminuted cannabis plant material is of a size greater than 0.3 millimeter and less than 4.2 millimeters;
(ii) the composition comprises at least 1% by weight moisture and less than 20% by weight moisture;
(iii) the cannabinoid concentration in a 1 gram fraction of at least 10 gram sample of said homogeneous composition, is within 15% of the cannabinoid concentration in a separate 1 gram fraction of said 10 gram sample;
(iv) optionally at least 70 weight % of the cannabis plant material in said composition is derived from cannabis flower; and
(v) optionally said homogeneous composition has bulk density greater than 0.02 grain/milliliter and less than 0.6 gram/milliliter.

48. A cigarette or a vaporizer cartridge containing the homogenous composition of claim 47.

49. A homogeneous cannabinoid-containing cannabis tablet, wherein
(i) said tablet moisture content is greater than 1% by weight and less than 20% by weight;
(ii) said tablet bulk density is at least 0.1 gram/milliliter and less than 1.2 gram/milliliter;
(iii) the cannabinoid concentration in a fraction of said tablet is within 15% of the cannabinoid concentration in a separate fraction of said tablet; and
(iv) optionally said tablet comprises at least 70% weight cannabis plant material.

50. A method for producing a homogeneous composition comprising comminuted cannabis plant material, comprising
(i) providing cannabis plant material comprising at least one cannabinoid and moisture, wherein said moisture content is less than 20 weight %;
(ii) maintaining said provided cannabis plant material at a temperature of less than 0° C. for at least 1 hour to form frozen plant material;
(iii) comminuting said frozen cannabis plant material by pressing said plant material against a mesh screen with a sieve size smaller than 4.2 millimeters to form an expelled homogeneous composition comprising comminuted cannabis plant material; and
(iv) collecting said expelled, homogeneous composition.

51. The method of claim 50, comprising providing a first cannabis plant material whereby a first collected expelled, homogeneous composition is formed, providing a second cannabis plant material whereby a second collected expelled, homogeneous composition is formed, and blending said first collected expelled, homogeneous composition with said second collected expelled, homogeneous composition to form a homogeneous blend composition.

52. The method of claim 51, further comprising analyzing a sample of said first collected expelled, homogeneous composition, analyzing a sample of said second collected expelled, homogeneous composition and blending said first composition and said second composition in a proportion calculated to form a homogeneous blend composition of pre-determined cannabinoid content.

53. The method of claim 50, wherein said homogeneous composition is characterized in that on sampling at least two separate 1 gram fractions of at least 10 gram sample of said homogeneous composition, the concentrations of said cannabinoid in the at least two 1 gram fractions are within 15% of each other.

54. The method of claim 50, wherein the bulk density of said homogeneous composition is greater than 0.02 gram/milliliter and less than 0.6 gram/milliliter.

55. A method of producing a cannabis cigarette, comprising (i) providing said homogeneous composition of claim 50; (ii) introducing said homogeneous composition into a feed vessel; (iii) providing a cigarette tube or a cigarette paper, wherein said provided cigarette tithe and/or said provided cigarette paper is optionally derived from hemp and wherein said provided cigarette tube is optionally cone shaped; and (iv) transferring said homogeneous composition from said feed vessel into said provided tube or onto said paper to form a cigarette, and wherein said transferring comprises vibrating the feed vessel at a rate of 0 to 200 vibrations per minute.

56. The method of claim 55, wherein said transferring said homogeneous composition from said feed vessel comprises transferring said homogeneous composition onto a tray and transferring said homogeneous composition from said tray into said tube or onto said paper.

57. The method of claim 56, wherein said homogeneous composition is transferred from said feed vessel to said tray via a screw conveyor.

58. The method of claim 55, further comprising blending said provided homogeneous composition with an additive, wherein said additive optionally comprises an additional cannabis plant material, to form a homogeneous mixture, which homogeneous mixture is transferred into said cigarette tube or onto said paper.

59. A cannabis cigarette produced according to the method of claim 55, wherein said cigarette contains a top fraction and a bottom fraction and the concentration of said cannabinoid in a 0.1 gram sample from the top fraction of the cigarette is within 15% of cannabinoid concentration in a 0.1 gram sample from the bottom fraction of the cigarette.

60. A batch of cannabis cigarettes produced according to the method of claim 55, wherein the content of said cannabinoid in one third of the batch of the cigarettes is within 15% of the cannabinoid content in a separate third of the batch of the cigarettes.

61. A method according to claim 50, further comprising (i) contacting said collected homogeneous cannabis composition with a liquid extractant for at least 1 minute to form cannabinoid-depleted plant material and a cannabinoid-enriched extractant, (ii) separating said cannabinoid-depleted plant material from said cannabinoid-enriched extractant, (iii) separating at least a fraction of said extractant from said cannabinoid-enriched extractant to form a cannabinoid enriched extract (iv) optionally wherein said cannabinoid-depleted plant material contains extractant and wherein said method optionally comprises separating extractant from said cannabinoid-depleted plant material to form desolventized cannabinoid-depleted plant material, and (v) optionally blending at least a fraction of said cannabinoid-depleted plant material and/or said desolventized cannabinoid-depleted plant material with at least a fraction of said collected homogeneous composition.

62. A method according to claim 50, further comprising pressing said collected homogeneous composition to form a homogeneous cannabis tablet.

63. A method according to claim 62, wherein the tablet has a bulk density of at least 0.1 gram/milliliter and less than 1.2 gram/milliliter.

64. A method according to claim 62, wherein the concentration of said cannabinoid in a fraction of said homogeneous cannabis tablet is within 15% of the cannabinoid concentration in a separate fraction of the tablet.

65. An apparatus for the production of cannabis cigarettes, comprising (i) a cigarette tubes holder, (ii) a feed vessel designed to contain a homogeneous composition comprising comminuted cannabis plant material and containing at least one opening, (iii) at least one first conveyor connected between said vessel opening and said holder and optionally (iv) a second conveyer designed to transfer said homogeneous composition from said vessel, through said opening, onto said first conveyor connected between said vessel and said first conveyor.

66. A homogeneous micronic composition comprising cannabinoid-containing comminuted cannabis plant material, wherein
(i) at least 70 weight % of the comminuted cannabis plant material is of a size less than 0.05 millimeter;
(ii) the composition comprises at least 1% by weight moisture and less than 20% by weight moisture;
(iii) the cannabinoid concentration in a 1 gram fraction of at least 10 gram sample of said homogeneous composition, is within 15% of the cannabinoid concentration in a separate 1 gram fraction of said 10 gram sample, and
(iv) optionally at least 50 weight % of the cannabinoid in said composition is a decarboxylated form.

67. A vaporizer cartridge containing said homogeneous micronic composition of claim 66.
